(19) United States
(12) Patent Application Publication
(10) Pub. No.: US 2014/0342048 A1
(43) Pub. Date: Nov. 20, 2014

(54) SAUCES, SPREADS, COOKING OILS, AND COOKING FATS MADE FROM VARIOUS INGREDIENTS

(71) Applicant: Alice Chang, South Pasadena, CA (US)

(72) Inventor: Alice Chang, South Pasadena, CA (US)

(21) Appl. No.: 14/090,891

(22) Filed: Nov. 26, 2013

Related U.S. Application Data
(60) Provisional application No. 61/823,314, filed on May 14, 2013.

(51) Int. Cl. A23D 7/005; A23L 1/39; A61K 31/439; A61K 36/06; A61K 36/062; A23D 9/007; A61K 38/47

(52) U.S. Cl. CPC A23D 7/0056; A23D 9/007; A23L 1/39; A61K 38/47; A61K 36/06; A61K 36/062; A61K 31/439
USPC 426/73; 426/589; 426/72; 426/108; 424/94.61; 424/195.16; 424/195.15; 514/305

(57) ABSTRACT

Sauces, spreads, cooking oils, and cooking fats are created from a wide range of various ingredients. Sauces, spreads, cooking oils, and cooking fats having at least one ingredient, with at least one flavoring, and at least one texture. At least one ingredient is selected from group including of at least one flavoring ingredient, at least one nutritional ingredient, at least one health ingredient, and at least one other ingredient. At least one ingredient is also created as small spherical balls as mixed in. Sauces can be served as appetizer and snack. And served as baby food, for children with less teeth, for old people, and for sick people. Sauces, spreads, cooking oils, and cooking fats are used for food preparation and food flavoring, including condiment and salad dressing. And used in manner selected from group consisting of room temperature, cold, warm, and hot.

### CROSS-REFERENCE TO RELATED APPLICATION

The present application is related to and claims priority from prior provisional application No. 61/823,314, filed May 14, 2013 and another prior provisional application No. 61/771,223, filed Mar. 1, 2013 which applications are incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of comestibles, and more specifically relates to sauces, spreads, cooking oils, and cooking fats made from various ingredients.

2. Description of the Related Art

Currently on the market, sauces, spreads, cooking oils, and cooking fats are very limited in the ingredients.

Various attempts have been made to solve the above-mentioned problems such as those found in U.S. Pat. Nos. 4,122,207, 5,260,083, 6599555, 8277858, 2004/0161522, 2013/0059029, 4578274, 5236732, 5607715, 7214400, 2002/0164413, 2007/0031579. This prior art is representative of comestibles relating to food. None of the above inventions and patents, taken either singly or in combination, is seen to describe the invention as claimed.

Ideally, sauces, spreads, cooking oils, and cooking fats are made from a wide range of various ingredients and yet manufactured at a modest expense.

### BRIEF SUMMARY OF THE INVENTION

In view of the foregoing disadvantages inherent in the known related comestibles art, the present invention provides sauces, spreads, cooking oils, and cooking fats made from various ingredients.

Sauces, spreads, cooking oils, and cooking fats comprising at least one ingredient, with at least one flavoring, and at least one texture. Sauces can be served as appetizer and snack. The sauces can be served as baby food, for children with less teeth, for old people, and for sick people. The sauces, spreads, cooking oils, and cooking fats are used for food preparation and food flavoring, including condiment and salad dressing. The sauces, spreads, cooking oils, and cooking fats are used in manner selected from the group consisting of room temperature, cold, warm, and hot. The sauces, spreads, cooking oils, and cooking fats can be created in various colors. The cooking oils are selected from the group consisting of fish oil, fish liver oil, vegetable oil, corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, watermelon seed oil, jojoba oil, and Diacylglycerol (DAG) oil. The cooking fats are selected from the group consisting of butter, margarine, cocoa butter, shea butter, ghee, clarified butter, vegetable fat, vegetable shortening, animal fat, lard, bacon fat, fatback, dripping, suet, tallow, chicken fat, duck fat, and schmaltz. At least one flavoring is selected from the group consisting of bland, sweet, sour, bitter, salty, spicy, pungent, umami, and metallic. At least one texture is selected from group consisting ofpowdery, soft, creamy, chewy, crunchy, crispy, jelly, gel, wax, liquid, oil, hard, semi solid, solid, and brittle.

The present invention holds significant improvements and serves as sauces, spreads, cooking oils, and cooking fats made from various ingredients. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### DETAILED DESCRIPTION

As discussed above, embodiments of the present invention relate to a comestible and more particularly to sauces, spreads, cooking oils, and cooking fats made from various ingredients.

As mentioned earlier, sauces, spreads, cooking oils, and cooking fats comprising at least one ingredient, with at least one flavoring, and at least one texture. Sauces can be served as appetizer and snack. The sauces can be served as baby food, for children with less teeth, for old people, and for sick people. The sauces, spreads, cooking oils, and cooking fats are used for food preparation and food flavoring, including condiment and salad dressing. The sauces, spreads, cooking oils, and cooking fats are used in manner selected from the group consisting of room temperature, cold, warm, and hot. The sauces, spreads, cooking oils, and cooking fats can be created in various colors. The cooking oils are selected from the group consisting of fish oil, fish liver oil, vegetable oil, corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, watermelon seed oil, jojoba oil, and Diacylglycerol (DAG) oil. The cooking fats are selected from the group consisting of butter, margarine, cocoa butter, shea butter, ghee, clarified butter, vegetable fat, vegetable shortening, animal fat, lard, bacon fat, fatback, dripping, suet, tallow, chicken fat, duck fat, and schmaltz. At least one flavoring is selected from group consisting of bland, sweet, sour, bitter, salty, spicy, pungent, umami, and metallic. At least one texture is selected from group consisting of powdery, soft, creamy, chewy, crunchy, crispy, jelly, gel, wax, liquid, oil, hard, semi solid, solid, and brittle.

At least one ingredient is selected from the group consisting of at least one flavoring ingredient, at least one nutritional ingredient, at least one health ingredient, and at least one other ingredient.

At least one ingredient is created as small spherical balls as mixed in selected from the group consisting of tapioca balls, sago pearls, starch balls, gelatin balls, and balls with both gelatin and starch. The spherical balls having a diameter of about 1 to 10 millimeters.

The small spherical balls having ingredient selected from the group consisting of gelatin and starch, including gelatin from fish and animal, beaten egg, egg white, pectin, vegetable gum, vegetable starch, corn starch, potato starch, sago, and tapioca. The gelatin from fish and animal can congeal below 15 degrees Celsius (59 degrees Fahrenheit). The small spherical balls with gelatin from fish and animal as ingredient can be served cold. The small spherical balls with starch, egg, and egg white as ingredient can be served cold, served at room temperature, and served reheated.

At least one ingredient is selected from the group consisting of caffeinated, and non caffeinated.

At least one ingredient is selected from the group consisting of diet, and non diet.

At least one flavoring ingredient is selected from the group consisting of natural flavor, artificial flavor, fruit flavor, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, cereal grain flavor, fruit skin flavor, vegetable skin flavor, vegetable flavor, vegetable oil flavor, vegetable fat flavor, meat flavor, seafood flavor, and other flavor. At least one flavoring ingredient is in form selected from the group consisting of skin, skinned, unskinned, unshelled, cut, uncut, extract, juice, sauce, liquid, oil, gel, jelly, wax, cream, paste, powder, thin layer, small particles, and small pieces. At least one flavoring ingredient can be uncooked, and cooked by method selected from the group consisting of deep frying, sun drying, roasting, baking, steaming, boiling, grilling, and frying.

For flavoring ingredient, other flavor is selected from the group consisting of artificial sweetener, salt, vinegar, sugar, syrup, honey, chocolate, coffee, tea, apple sauce, raisin, cream, sour cream, milk, ice cream, yogurt, gelato, sherbet, cheese, cream cheese, cottage cheese, mozzarella, mascarpone, ricotta, butter, margarine, royal jelly, caramel, butterscotch, peanut butter, cookie, biscuit, cake, jelly, pudding, custard, taco, bread, pita, tortilla, pancake, crepe, pastry, waffle, donut, pie, roll, candy, marshmallow, fish oil, fish liver oil, vegetable shortening, plant wax, animal wax, beeswax, carnauba wax, tonic water, glycerol, egg, and malt.

For flavoring ingredient, fruit flavor and fruit skin flavor are selected from the group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, marula, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine.

For flavoring ingredient, vegetable flavor, vegetable skin flavor, vegetable oil flavor, and vegetable fat flavor are selected from the group consisting of root vegetable, fruit vegetable, flower bud, flower, stem shoot, stem, leaf, bulb, cereal grain, nut, seed, bean, sea vegetable, and other types of vegetable, including aloe vera, jojoba, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turmeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, and bean sprout.

For flavoring ingredient, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, and cereal grain flavor are selected from the group consisting of leaf, flower, flower bud, pollen, bulb, root, stem, stem shoot, and herb, including hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litseacubeba, marjoram, melaleuca, mountain savory, mugwort, myrrh, myrtle, neroli, nutmeg, orris, palosanto, petitgrain, pine, ravensara, red cedar, rosewood, sage, sassafras, schisandra, spikenard, spruce, tea tree, tsuga, valerian, yarrow, ylangylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff.

For flavoring ingredient, vegetable oil flavor is selected from the group consisting of corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, jojoba oil, and watermelon seed oil.

For flavoring ingredient, meat flavor is selected from the group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood.

For flavoring ingredient, seafood flavor is selected from the group consisting of squid, cuttle fish, shrimp, crab, crayfish, octopus, clam, oyster, scallop, lobster, abalone, conch, jellyfish, prawn, hill, mussel, sea cucumber, sea urchin, eel, snail, shrimp roe, crab roe, lobster roe, fish roe, and fish.

At least one nutritional ingredient comprises essential nutrient selected from the group consisting of vitamin, dietary mineral, amino acid, and fatty acid. The vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K. The dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium. The amino acid includes histidine, isoleucine, leucine, lysine, methionine, cysteine, phenylalanine, tyrosine, threonine, tryptophan, and valine. The fatty acid includes alpha-linoleic acid, and linoleic acid. At least one nutritional ingredient is in form selected from the group consisting of extract, juice, sauce, liquid, oil, gel, jelly, wax, cream, paste, powder, thin layer, small particles, and small pieces.

At least one health ingredient comprises medication and other health ingredient. The medication is mixed into the food substance for ease of consumption. The other health ingredient is selected from the group consisting of electrolyte, lactase, yeast, propolis, and quinine. At least one health ingredient is in form selected from the group consisting of extract, juice, sauce, liquid, oil, gel, jelly, wax, cream, paste, powder, thin layer, small particles, and small pieces.

At least one other ingredient comprises food additive selected from the group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

At least one ingredient further comprises at least one binding agent which is an edible binder and is selected from the group consisting of protein component and starch component. The protein component is selected from the group consisting of gelatin, beaten egg, and egg white. The starch component is selected from the group consisting of vegetable gum, vegetable starch, and vegetable flour. The vegetable gum is selected from the group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum. The vegetable starch and vegetable flour are selected from the group consisting of root vegetable, cereal grain, nut, seed, bean, pectin, sago, and tapioca. At least one binding agent can be cooked by method selected from the group consisting of mixing with water, boiling, and steaming. The gelatin from fish and animal congeals below 15 degrees Celsius (59 degrees Fahrenheit).

At least one ingredient with at least one binding agent comprising gelatin can be refrigerated to serve cold but not reheated because gelatin from fish and animal congeals below 15 degrees Celsius (59 degrees Fahrenheit). At least one ingredient with at least one binding agent selected from the group consisting of beaten egg, egg white, and starch component can be served at room temperature, refrigerated to serve cold, and reheated to serve hot.

The sauces, spreads, cooking oils, and cooking fats having packaging as individual package and multiple package. The packaging can be in air tight container. The packaging is selected from the group consisting of paper, plastic, metal, and glass.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent set forth in the appended claims:

1. Sauces, spreads, cooking oils, and cooking fats comprising:
at least one ingredient, with
at least one flavoring, and
at least one texture;
wherein said sauces can be served as appetizer and snack;
wherein said sauces can be served as baby food, for children with less teeth, for old people, and for sick people;
wherein said sauces, spreads, cooking oils, and cooking fats are used for food preparation and food flavoring, including condiment and salad dressing;
wherein said sauces, spreads, cooking oils, and cooking fats are used in manner selected from group consisting of room temperature, cold, warm, and hot;
wherein said sauces, spreads, cooking oils, and cooking fats can be created in various colors;
wherein said cooking oils are selected from group consisting of fish oil, fish liver oil, cod liver oil, shark liver oil, vegetable oil, corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, marula oil, mongongo nut oil, mustard oil, pecan oil, perilla oil, pine nut oil, pistachio oil, poppyseed oil, rapeseed oil, watermelon seed oil, jojoba oil, and Diacylglycerol oil;
wherein said cooking fats are selected from group consisting of butter, margarine, cocoa butter, shea butter, ghee, clarified butter, vegetable fat, vegetable shortening, animal fat, lard, bacon fat, fatback, dripping, suet, tallow, chicken fat, duck fat, and schmaltz;
wherein said at least one flavoring is selected from group consisting of bland, sweet, sour, bitter, salty, spicy, pungent, umami, and metallic; and
wherein said at least one texture is selected from group consisting of powdery, soft, creamy, chewy, crunchy, crispy, jelly, gel, wax, liquid, oil, hard, semi solid, solid, and brittle.

2. The sauces, spreads, cooking oils, and cooking fats of claim 1 wherein said at least one ingredient is selected from group consisting of at least one flavoring ingredient, at least one nutritional ingredient, at least one health ingredient, and at least one other ingredient.

3. The sauces, spreads, cooking oils, and cooking fats of claim 1 wherein said at least one ingredient is created as small spherical balls as mixed in selected from group consisting of tapioca balls, sago pearls, starch balls, gelatin balls, and balls with both gelatin and starch; and said spherical balls having a diameter of about 1 to 10 millimeters.

4. The sauces, spreads, cooking oils, and cooking fats of claim 3 wherein said small spherical balls having ingredient selected from group consisting of gelatin and starch, including gelatin from fish and animal, beaten egg, egg white, pectin, vegetable gum, vegetable starch, corn starch, potato starch, sago, and tapioca; said gelatin from fish and animal can congeal below 15 degrees Celsius; said small spherical balls with said gelatin from fish and animal as ingredient can be served cold; and said small spherical balls with said starch, said egg, and said egg white as ingredient can be served cold, served at room temperature, and served reheated.

5. The sauces, spreads, cooking oils, and cooking fats of claim 1 wherein said at least one ingredient is selected from group consisting of caffeinated, and non caffeinated.

6. The sauces, spreads, cooking oils, and cooking fats of claim 1 wherein said at least one ingredient is selected from group consisting of diet, and non diet.

7. The sauces, spreads, cooking oils, and cooking fats of claim 2 wherein said at least one flavoring ingredient is selected from group consisting of natural flavor, artificial flavor, fruit flavor, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, cereal grain flavor, fruit skin flavor, vegetable skin flavor, vegetable flavor, vegetable oil flavor, vegetable fat flavor, meat flavor, seafood flavor, and other flavor; said at least one flavoring ingredient is in form selected from group consisting of skin, skinned, unskinned, unshelled, cut, uncut, extract, juice, sauce, liquid, oil, gel, jelly, wax, cream, paste, powder, thin layer, small particles, and small pieces; and said at least one flavoring ingredient can be uncooked, and cooked by method selected from group consisting of deep frying, sun drying, roasting, baking, steaming, boiling, grilling, and frying.

8. The sauces, spreads, cooking oils, and cooking fats of claim 7 wherein said other flavor is selected from group consisting of artificial sweetener, salt, vinegar, sugar, syrup, honey, chocolate, coffee, tea, apple sauce, raisin, cream, sour cream, milk, ice cream, yogurt, gelato, sherbet, cheese, cream cheese, cottage cheese, mozzarella, mascarpone, ricotta, butter, margarine, royal jelly, caramel, butterscotch, peanut butter, cookie, biscuit, cake, jelly, pudding, custard, taco, bread, pita, tortilla, pancake, crepe, pastry, waffle, donut, pie, roll, candy, marshmallow, fish oil, fish liver oil, vegetable shortening, plant wax, animal wax, beeswax, carnauba wax, tonic water, glycerol, egg, and malt.

9. The sauces, spreads, cooking oils, and cooking fats of claim 7 wherein said fruit flavor and fruit skin flavor are selected from group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine.

10. The sauces, spreads, cooking oils, and cooking fats of claim 7 wherein said vegetable flavor, vegetable skin flavor, vegetable oil flavor, and vegetable fat flavor are selected from group consisting of root vegetable, fruit vegetable, flower bud, flower, stem shoot, stem, leaf, bulb, cereal grain, nut, seed, bean, sea vegetable, and other types of vegetable, including aloe vera, jojoba, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turmeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, and bean sprout.

11. The sauces, spreads, cooking oils, and cooking fats of claim 7 wherein said plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, and cereal grain flavor are selected from group consisting of leaf, flower, flower bud, pollen, bulb, root, stem, stem shoot, and herb, including hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litseacubeba, marjoram, melaleuca, mountain savory, mugwort, myrrh, myrtle, neroli, nutmeg, orris, palosanto, petitgrain, pine, ravensara, red cedar, rosewood, sage, sassafras, schisandra, spikenard, spruce, tea tree, tsuga, valerian, yarrow, ylangylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, marula, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortirio, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff.

12. The sauces, spreads, cooking oils, and cooking fats of claim 7 wherein said vegetable oil flavor is selected from group consisting of corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, jojoba oil, and watermelon seed oil.

13. The sauces, spreads, cooking oils, and cooking fats of claim 7 wherein said meat flavor is selected from group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood.

14. The sauces, spreads, cooking oils, and cooking fats of claim 7 wherein said seafood flavor is selected from group consisting of squid, cuttle fish, shrimp, crab, crayfish, octopus, clam, oyster, scallop, lobster, abalone, conch, jellyfish, prawn, krill, mussel, sea cucumber, sea urchin, eel, snail, shrimp roe, crab roe, lobster roe, fish roe, and fish.

15. The sauces, spreads, cooking oils, and cooking fats of claim 2 wherein said at least one nutritional ingredient comprises essential nutrient selected from group consisting of vitamin, dietary mineral, amino acid, and fatty acid; said vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K; said dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium; said amino acid includes histidine, isoleucine, leucine, lysine, methionine, cysteine, phenylalanine, tyrosine, threonine, tryptophan, and valine; said fatty acid includes alpha-linoleic acid, and linoleic acid; and said at least one nutritional ingredient is in form selected from group consisting of extract, juice, sauce, liquid, oil, gel, jelly, wax, cream, paste, powder, thin layer, small particles, and small pieces.

16. The sauces, spreads, cooking oils, and cooking fats of claim 2 wherein said at least one health ingredient comprises medication and other health ingredient; said medication is mixed into said food substance for ease of consumption; said other health ingredient is selected from group consisting of electrolyte, lactase, yeast, propolis, and quinine; and said at least one health ingredient is in form selected from group consisting of extract, juice, sauce, liquid, oil, gel, jelly, wax, cream, paste, powder, thin layer, small particles, and small pieces.

17. The sauces, spreads, cooking oils, and cooking fats of claim 2 wherein said at least one other ingredient comprises food additive selected from group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

18. The sauces, spreads, cooking oils, and cooking fats of claim 1 wherein said at least one ingredient further comprises at least one binding agent which is an edible binder and is selected from group consisting of protein component and starch component; said protein component is selected from group consisting of gelatin, beaten egg, and egg white; said starch component is selected from group consisting of vegetable gum, vegetable starch, and vegetable flour; said vegetable gum is selected from group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum; said vegetable starch and vegetable flour are selected from group consisting of root vegetable, cereal grain, nut, seed, bean, pectin, sago, and tapioca; said at least one binding agent can be cooked by method selected from group consisting of mixing with water, boiling, and steaming; and said gelatin from fish and animal congeals below 15 degrees Celsius.

19. The sauces, spreads, cooking oils, and cooking fats of claim 1 wherein said at least one ingredient with at least one binding agent comprising gelatin can be refrigerated to serve cold but not reheated because gelatin from fish and animal congeals below 15 degrees Celsius; and said at least one ingredient with at least one binding agent selected from group consisting of beaten egg, egg white, and starch component can be served at room temperature, refrigerated to serve cold, and reheated to serve hot.

20. The sauces, spreads, cooking oils, and cooking fats of claim 1 having packaging as individual package and multiple package; said packaging is in air tight container; and said packaging is selected from group consisting of paper, plastic, metal, and glass.
